(19) United States
(12) Patent Application Publication
(10) Pub. No.: US 2010/0215769 A1
(43) Pub. Date: Aug. 26, 2010

(54) Natural occurring nutrient supplement compositions and methods of treatment using same by improving resistance to DNA damage, enhancing DNA repair and stimulating immune function wherein metabolic competition is avoided

(76) Inventors: Ronald W. Pero, (US); Stuart Garret, (US)

Correspondence Address: Stuart Garret;Ronald W. Pero, 27 Ambassador Circle, Rancho Mirage, CA, 92270, US

(21) Appl. No.: 12/391,431

(22) Filed: Feb. 24, 2009

(51) Int. Cl. A61K 33/24; A61P 3/02

(52) U.S. Cl. 424/643

(57) ABSTRACT

Selectively administering to humans, as a daily dosage, a combination of resveratrol-carotenoids, nicotinamide (or niacin or a precursor thereof) and a source of zinc, in excess of normal dietary levels, for improving resistance to DNA damage, enhancing DNA repair capacity, and stimulating immune function.

### CROSS REFERENCE RELATED PATENT BY RONALD W. PERO & STUART GARRET

This application claims benefit of U.S. Pat. No. 6,020,351 issued Feb. 1, 2000 invented by Ronald W. Pero and later abandoned after issuance. The knowledge in that patent is included in its entirety herein by this reference.

### BACKGROUND OF THE INVENTION

This invention relates to novel and improved compositions for and methods of treating humans and other animals to reduce DNA damage, enhance DNA repair capacity, and stimulate immune cellular function. More particularly, it relates to the administration to human (or other animal) subjects of a combination of resveratrol material, carotenoid material, nicotinamide material, and zinc source material (all as hereinafter defined), e.g. as a drug treatment or a daily dietary supplement, and to compositions containing that combination of materials.

The term “carotenoid material” as used herein means carotenoids, such as material selected from the group consisting of alpha carotene, beta-carotene, canthaxanthin, lycopene and mixtures thereof. The term “nicotinamide material” as used herein means material selected from the group consisting of nicotinamide, niacin, tryptophan (an amino acid precursor to niacin synthesis) and mixtures thereof. The term “zinc source material” as used herein means an appropriate source of zinc for administration to humans and/or other animals, e.g. one or more zinc salts, such as zinc sulfate or other zinc salts like amino acids such as methionine or aspartate, dipeptides, gluconates, halides, nitrates, oxides or acetates. These ingredients have been fully described for use treating patients having increased DNA damage and decreased DNA repair leading to an impaired immune function in U.S. Pat. No. 6,020,351, and in Yezhou Sheng, D.M.Sc., Ronald W. Pero, Ph.D., Anders R. Olsson, Ph.D., Carl Bryngelsson, B.Sc., and Jianyi Hua, M.D DNA Repair Enhancement by a Combined Supplement of Carotenoids, Nicotinamide, and Zinc. Cancer Detection and Prevention 1998; 22(4):284-292.

Resveratrol material is defined herein as a polyphenol (e.g. 3,5,4′-trihydroxy-stilbene) that can induce cell cycle arrest thereby permitting greater opportunities to remove DNA damage by DNA repair, and thus stimulate normal cell survival and longevity, which in turn can also result in an increased immune cell function (Dario R. Valenzano et al. Current Biology 16 (3): 296-300, 2006; Feng Y H et al. Acta Pharmacol Sin 2002 10: 893-897, 2002; Susanne Andrea Gatz et al. Carcinogenesis 2008 29(3):519-527). These properties for resveratrol materials were not known at the time carotenoid-nicotinamide-zinc materials were described for their non-competitive modes of action in 2000 that directly related to the mechanisms of DNA damage and its repair in U.S. Pat. No. 6,020,151. Thus, this is a novel discovery adding to the previous scientific knowledge already cited. In a specific aspect, the invention relates to an improved and novel combination of naturally occurring resveratrol material, carotenoid material, nicotinamide material and zinc source material as a combined treatment to aid patients in resisting cellular DNA damage by reducing oxidative cellular damage through enhancing cellular DNA repair capacity (and stimulating immune cellular function) via different modes of action; e.g. cell cycle arrest (resveratrol), oxidative radical scavenger (carotenoids), co-substrate enzymatic stimulator (NAD via nicotinamide), and co-factor repair enzyme activator (Poly ADP ribose polymerase, PARP, the DNA binding zinc finger DNA binding region). In another specific sense, this combination of chemicals can be used as a dietary supplementation, or as a drug treatment, to prevent (or improve an individual's ability to resist) DNA damage, enhance DNA repair and stimulate immune function in diseases where these processes are central to the manifested disease state; e.g. ageing, cancer, cardiovascular disease and autoimmune disorders such as diabetes, rheumatoid arthritis and ulcerative colitis (Cross et al., Ann. Int. Med. 107:526-545, 1987; Harris, C. C., Cancer Res. 51(Suppl): 5023s-5044s, 1991; Olin, K. L. et al., Proc. Soc. Expt. Biol and Med. 203(4):461-466, 1993).

Humans have been selected over hundreds of thousands of years to respond to not one chemical but to a myriad of chemicals coming to us through our environment mainly thorough the diet. One can assume that our physiology is extremely well balanced to handle and process these chemical mixtures to extract as efficiently as possible the necessities of life such as nutritional energy sources and chemicals to maintain cellular homeostasis and reproduction. This has to be accomplished without introducing any toxicological consequences. Hence, it follows there is a reasonable likelihood that when humans see natural medicines above the levels normally found in the diet or environment, there exists a strong interaction between the mega doses of natural medicines, so that one supplement limits the uptake and metabolism of another, in an effort to provide a natural selection model by which humans can be protected from the toxicological consequences of overdosing. For example, the practice of prior art teaches that carotenoids and vitamins E or C are all radical (electrophilic) scavengers and that these natural products can be combined into supplements for additive biological effects. However, recent literature has not confirmed this practice based on scientific assumption because it was shown that these radical scavengers could inhibit each other's uptake and negate the desired induction of biological effects (Inform 6(7):778-783, 1995; Zhang et al., J. Clin. Nutr. 62:1477S-1482S, 1995; Niki et al., Am. J. Clin. Nutr. 62:1322S-1326S, 1995).

However, various commercial products so far introduced do not establish or make obvious that the any specific combinations of resveratrol, carotenoids, nicotinamide and zinc are effective at reducing cellular DNA damage induction or enhancing DNA repair and immune function, when or if other ingredients are also included that have similar modes of action. On the contrary, as demonstrated in U.S. Pat. No. 6,020,351 previously by applicant, it was found that the administration of carotenoids, nicotinamide and zinc in combination with other natural medicines or nutrients such did not reduce cellular DNA damage induction or enhance DNA repair and immune function as had been assumed but not proven in the prior art. This discovery was also consistent with the prior art (Inform 6(7):778-783, 1995; Zhang et al., J. Clin. Nutr. 62:1477S-1482S, 1995; Nidi et al., Am. J. Clin. Nutr. 62:1322S-1326S, 1995) which has confirmed that the natural products (e.g. medicines or nutrients) having similar modes of biochemical action have been shown to block each other's uptake and absorption, thus resulting in altered biological functions. It follows then although not practiced in the prior art that it cannot be assumed supplementing an a priori combination of natural products above dietary levels will result in additive biological effects of each product administered separately without previously establishing they possess different modes of action to bring about a common biological effect such as DNA repair enhancement. For example, here we describe a the novel combination of resveratrol material, carotenoid material, nicotinamide material and a zinc source all of which have been shown to enhance DNA repair and reduce DNA damage by different molecular mechanisms, thus permitting a combination of natural products having a non-competitive activation of DNA repair having potent anti-aging properties.

It was not obvious or practiced in the prior art that reservatrol could be combined with other nutritional agents known to enhance DNA repair by avoiding metabolic competition. For example, even though it has been known since 1998, there have been no reports in the literature that resveratrol material also known to be a DNA repair enhancer could be combined with carotenoid material, nicotinamide material, or zinc material to create supplements that enhance DNA repair by avoiding metabolic competition. Hence it was not obvious to one skilled in the art that resveratrol could be combined with carotenoids, nicotinamide or Zinc salts to enhance DNA re, because they had different mechanisms of DNA repair enhancement than did resveratrol.

Known molecular mechanisms of resveratrol material. The main effects of resveratrol are to regulate cell cycle events that favor growth arrest allowing DNA repair enhancement before cells die from DNA damage blockage of cell replication (Dario R. Valenzano et al. Current Biology 16 (3): 296-300, 2006; Feng Y H et al. Acta Pharmacol Sin 2002 10: 893-897, 2002; Susanne Andrea Gatz et al. Carcinogenesis 2008 29(3): 519-527; Feng Y H et al. Acta Pharmacol Sin 23(10): 893-897, 2002; Loma Whyte et al. Cancer Res 67(24):12007-17, 2007). There changes in both gene and protein expression, such as the up-regulation of p53 and p21 and the down-regulation of cyclin A, chk1, CDC27, and Eg5 (a mitotic motor protein). Resveratrol also alters the intracellular Smad signaling of the TGF-β pathway. Finally, dietary restriction, the best-studied life-extension treatment, causes over expression of SIRT1 (H. Y. Cohen et al. Science 305 pp. 390-392, 2004), and since these effects are not additive to resveratrol they suggest that a similar molecular mechanism to dietary restriction.

Known molecular mechanisms of carotenoid material. The exact mechanism of action of carotenoids such as beta carotene is not fully understood but it is commonly accepted scientifically that one primary mechanism is to directly scavenge oxygen derived free radicals produced either as by-products of metabolism or from exogenous environmental exposures (Lieber, D. C., Ann. N. Y. Acad. Sci. 691:20-31, 1993; Bohm, F. et al., J. Photochem. Photobiol. 21(2-3):219-221, 1993; Regnault, C. et al., Ann. Pharmacotherapy 27(11):1349-1350, 1993). As a free radical scavenger, carotenoids can be expected to reduce or protect against the chemical damage induced in DNA, RNA and protein of cells by toxic environmental exposures or endogenous cellular metabolic errors that ultimately can result in a disease state. On the other hand, nicotinamide and zinc salts do not possess this chemical property, which results in an improved biological cellular function.

Known molecular mechanisms of nicotinamide material. Nicotinamide and its metabolic equivalent nicotinic acid (niacin, vitamin B) or even tryptophan, which is the synthetic precursor to niacin, is the main precursor for the formation and maintenance of the cellular pool of NAD (Bemofsky, Mol. Cell. Biochem. 33:135-143, 1980; Olsson, A. et al., Biochem. Pharmacol. 45:1191-1200, 1993). NAD is essential for cellular ATP production and maintenance of the cell's redox potential, and it is also the substrate for the DNA repair enzyme, poly ADP-ribosyl transferase (ADPRT). Niacin deprivation decreases the NAD pools significantly both in tissue culture cells (Jacobson, E. et al., IN: ADP-Ribosylation Reactions (Poirier, G. G. and Moreau, P., eds.), pp. 153-162, Springer Verlag, New York, N.Y., 1992), and animal systems (Zhang et al., J. Nutri. 123:1349-1355, 1993) as well as humans (Fu et al., J. Nutri. 119:1949-1955, 1989). The depleted cells have an increased sensitivity to DNA damage and the levels of poly (ADP-ribose) production in cultured cells (Jacobson, E. L., as cited, 1992) or in rat liver (Rawling et al., J. Nutri. 124:1597-1603, 1994) were significantly lower after mild nicotinamide deficiency. On the other hand, when niacin was given as a supplement to ordinary nutrition (i.e. above known dietary levels) the NAD pool increased and the cells were less sensitive to oxygen radicals (Weinberg, A. B., Mutational Res. 216:197-201, 1989). Therefore, it is obvious from this review of the prior art that the primary mechanism of action of nicotinamide/niacin differs from carotenoids and zinc in that the cell's potential for energy metabolism is increased by amplifying NAD and ATP pool supplies (i.e. these biochemical's are the energy sources of living organisms) which in turn is useful to cells, tissues and organs to reduce DNA damage, enhance DNA repair (i.e. poly ADP-ribosylation) and stimulate immune function where the relevance to the disease state is apparent (Pero, R. W. et al., Biochimie 77:385-393, 1995).

Known molecular mechanisms of zinc source material. Zinc differs from the reservatrol, carotenoids and nicotinamide with regard to its mechanism of action in that it influences disease development and immune function by being an essential co-factor in several enzyme functions involving replication, DNA repair and antioxidant defense of cells. Zinc is required for cell replication and DNA polymerase activity (Williams, R. O. et al., J. Cell Biol. 58:594-601, 1973). There are two zinc fingers in the DNA binding domain of the poly adenosine diphosphate ribosyl transferase (ADPRT) gene and other DNA repair proteins (Dawat, P. et al., Microbiol. 141 (Pt 2):411-417, 1995; Matsuda, T. et al., J. Biol. Chem. 270(8):4152-4157, 1995; Chiriccolo, M. et al., Mutation Res. 295(3):105-111, 1993) which contain cysteine residues (i.e. an amino acid), and if these cysteine residues are oxidized at their thiol constituents, they would prevent DNA binding and participation in DNA repair (Mazen et al., Nucleic Acid Res. 17:4689-4698, 1989; de Murcia, G. et al., BioEssays 13(9):455-462, 1989; Pero, R. W. et al., Biochimie 77:385-393, 1995; Althaus, F. et al., Mol. Cell. Biochem. 138(1-2):53-59, 1994). Moreover, super oxide dismutase is an antioxidant enzyme protecting cells from the harmful super oxide anion because this radical is a substrate for the enzymatic reaction that also requires zinc as a cofactor (Brunori, M. and Rotilio, G., Methods in Enzymology 105:22-35, 1984).

In summation, even though resveratrol, carotenoids, nicotinamide/niacin and zinc have been shown to have some enabling utility in cell and animal models as single agents in the prevention of certain diseases and in the stimulation of immune function, there has been a lack of corresponding, consistent data in humans (Bodgen, J. D. et al., Amer. J. Clin. Nutri. 48:655-663, 1988; Walsh, C. T. et al., Environmental Health Perspectives 102(Suppl. 2):5-46, 1994; Dario R. Valenzano et al. Current Biology 16 (3):296-300, 2006). In addition, it is not possible for one skilled in the art to a priori predict whether agents with different mechanisms of action will be synergistic or additive to the biological response they will elicit when given in combination. However, the prior art as described and shown in U.S. Pat. No. 6,202,351 does teach that non-competitive molecular mechanisms that enhance DNA repair will at least be additive to each other.

### SUMMARY OF THE INVENTION

The present invention, in a first aspect, broadly contemplates the provision of a composition of matter for administration to humans or other animals “consisting essentially of” a combination of resveratrol material, carotenoid material, nicotinamide material and zinc source material and “essentially free of other bioactive nutrient agents” which may compete with their known modes of action. The term “bioactive nutrient agents” is employed herein as a generic designation for vitamins, minerals and other bioactive substances serving as anti-oxidants, anti-oxidant co-factors, or otherwise contributing to disease prevention, inhibition of DNA damage, improvement of DNA repair capacity, and/or enhanced immune function, such as have heretofore been sold in concentrated, isolated, or combined form as dietary supplements and the like for human and/or animal consumption.

Stated in other words, then, the invention in this aspect embraces compositions containing resveratrol material, carotenoid material, nicotinamide material and zinc source material, and no other active nutrient agents having competing modes of action to these specified agents. The compositions of the invention may be embodied in formulations for oral administration, or alternatively, in formulations for parenteral administration.

In illustrative or preferred practice of the invention, the carotenoid material may be selected from the group consisting of resveratrol (3,5,4′-trihydroxy-stilbene or equivalent polyphenol); alpha carotene, beta carotene, canthaxanthin, lycopene and mixtures thereof; the nicotinamide material may be selected from the group consisting of nicotinamide, niacin, tryptophan and mixtures thereof; and the zinc source material may be one or more zinc salts.

For human administration, the resveratrol material, carotenoid material, nicotinamide material and zinc source material may be present in proportions effective, in combination, to improve resistance to DNA damage, enhance DNA repair capacity, and stimulate immune function in a human subject to whom the composition is administered as a daily dosage.

The invention also contemplates the provision of a method of treating a human or other animal subject, consisting of administering resveratrol material, carotenoid material, nicotinamide material and zinc source material to the subject to selectively supplement the subject's dietary intake thereof (i.e., without supplementing the dietary intake of any other active nutrient agents having competing modes of action) and repeating the administration on a substantially daily basis.

Thus, in a particular sense, the invention contemplates the provision of a method of treating a human subject consisting of selectively administering to the subject resveratrol material, carotenoid material, nicotinamide material and zinc source material in daily dosage amounts effective, in combination, to improve resistance to DNA damage, enhance DNA repair capacity, and stimulate immune function. In a specific example of currently preferred dosage range for humans, about 1500-6000 mg resveratol material, about 100 mg of carotenoid material, about 100 mg of nicotinamide material and about 10 mg of zinc source material are administered daily in this method.

From a theoretical standpoint, this invention is based on a principle of combining chemical products which are individually known to possess either cancer preventive or immune stimulatory properties into one formulation which contains only active components where at least one mechanism of action for each active component is known to be different from the known mechanisms of action of the other components to stimulate DNA repair. So far as applicant is aware, this particular combination of ingredients has not heretofore been recognized in the art.

The invention involves the discovery that natural products should not be combined into a natural medicine unless one tests whether each ingredient is additive to the overall desired biological effect, and that one way to accomplish this endpoint is to not combine natural products that have similar modes of action and thus competitive routes of absorption and excretion without first testing the combination for additive effects. That is to say, the present invention avoids inhibited uptake and absorption of natural products, thereby obtaining additive biological effects, by combining only natural products having well defined different and thus potentially non-competitive modes of action which is, for example, the case with the exclusive combination of carotenoids+nicotinamide+zinc.

The practice of this invention involves supplementing humans or animals for example, by the oral, intraperitoneal, intravenous, subcutaneous or intramuscular routes of administration with the combination of resveratrol+carotenoids+nicotinamide/niacin+an appropriate zinc salt at a dose of this combination that exceeds a normal dietary supplementation. The practice of the prior art teaches that dietary supplementation containing this combination together with simultaneous supplementation of other nutrients and/or natural products cannot enhance immune function (Payette, H. et al., Am. J. Clin. Nutr. 52:927-932, 1990; Zhang et al., J. Clin. Nutr. 62:1477S-1482S, 1995; U.S. Pat. No. 6,020,351) but when resveratrol, carotenoids (as Caroplex, C. E. Jamieson, Ltd., Ontario, Canada), nicotinamide and a zinc salt are given alone in the absence of other natural supplements above dietary levels, e.g. 500-1500 mg, 100 mg, 100 mg and 10 mg by oral daily administration, respectively, the resistance to oxidative cellular DNA damage, and enhancement of DNA repair and immune function were observed.

The clinical evaluation in a previous study (U.S. Pat. No. 6,020,351) was determined by comparing each individual's biological response before and after supplementation. In such a manner, each individual became his own control; e.g. the male subjects were given baseline measurements of resistance to cellular DNA damage, enhancement of DNA repair and stimulation of immune function once a week for 4 weeks, and then they were supplemented daily and the same measurements repeated once a week for the last 5 weeks of a 7 week intervention period. The before measurements (i.e. n=4) were the baseline biological response parameters to be compared to the after measurements (i.e. n=5). One individual was not supplemented to provide a control for the supplemented individuals. The data from this experimental design has taught that resistance to cellular DNA damage, enhancement of DNA repair and stimulation of immune function were all significantly modulated by a combination of carotenoids+nicotinamide+zinc when administered as an exclusive drug combination above dietary levels, but not when co-administered together with other additional nutrient or natural product supplements.

### DETAILED DESCRIPTION

In the specific embodiment hereinafter described in detail, this invention involves the use of a combination consisting essentially of resveratrol+carotenoid+nicotinamide+zinc gluconate (and no other active nutrient agents with competing molecular mechanisms) as an oral supplement, over and above the normal levels of these components in the diet, administered daily to increase an individual's resistance to cellular DNA damage, enhance cellular DNA repair and stimulate immune cell responsiveness in vivo. The design of the study to prove this invention was based on combining substances with known properties to prevent cancer and stimulate immune function but with differing mechanisms of action; e.g. resveratrol=cell cycle modulation, carotenoids=electrophilic scavenger of radicals produced endogenously by cells or exogenously by the environment, nicotinamide=amplified source of energy via increased production of NAD or ATP, and zinc=an essential cofactor to antioxidant, replicative and DNA repair enzymes in cells. The hypothesis was that since none of these substances have produced consistent effects in humans as a single administered agent, this shortcoming could be overcome when administered in combination because these substances might produce a consistently additive or synergistic chemo-preventive biological response because of non-competitive modes of action instead of, for example, an inhibited one.

### Detailed Analysis of Prior Art for Nutra-Resveratrol Patent Application

To our knowledge, there are 3 public-disclosures published that require further analysis to explain as to why they are not prior art to the proposed new Nutra-Resveratrol patent application. All three disclosures get this distinction because they propose combining natural ingredients that can enhance anti-aging effects by combining them in a mixture to reduce accumulation of cellular DNA damage by enhancing DNA repair. Whereas there are many patents professing that compositions of ingredients have widespread health benefits, there are no others stated that the mechanism is by reducing DNA damage by enhancing biological removal of it by the process of cellular DNA repair. This potential prior art is found in:
1. U.S. Pat. No. 6,020,351 (granted, abandoned)
2. US patent application 20050196467 (Ser. No. 11/106,200) (deuterium abandoned)
3. US patent application 20050287227 (Ser. No. 11/046,108) (uncaria abandoned)
1. U.S. Pat. No. 6,020,351 was issued Feb. 1, 2000 and is the most significant prior art to the Nutra-Resveratrol application. This invention clearly teaches the only established way to produce compositions of natural occurring ingredients to enhance anti-aging effects by enhancing DNA repair mechanisms is stated therein as: “The invention involves the discovery that natural products should not be combined into a natural medicine unless one tests whether each ingredient is additive to the overall desired biological effect, and that one way to accomplish this endpoint is to not combine natural products that have similar modes of action and thus competitive routes of absorption and excretion without first testing the combination for additive effects. That is to say, the present invention avoids inhibited uptake and absorption of natural products, thereby obtaining additive biological effects, by combining only natural products having well defined different and thus potentially non-competitive modes of action which is, for example, the case with the exclusive combination of carotenoids+nicotinamide+zinc.” There is no other prior art existing to this concept for one skilled in the art to produce anti-aging effects with compositions that enhance DNA repair. In other words, this patent contains the data; knowledge and analysis that teach compositions of DNA repair ingredients that are synergistic (or are additive) to each other will be identified only if each ingredient has a different molecular mechanism by which it enhances DNA repair. Hence knowledge of the primary mechanism of DNA repair enhancement is critical to producing a composition that would indeed enhance DNA repair permitting a parallel enhancement of anti-aging effects. Moreover, the primary claim of a combination of carotenoid material, nicotinamide material and zinc source material and “essentially free of other active ingredients” for treating humans and animals limits the claim to only these three specific ingredients without knowing the status of metabolic competition from any other ingredients desired to be added.

Whereas U.S. Pat. No. 6,020,351 is prior art only for carotenoids+nicotinamide+zinc, it is not when other DNA repair ingredients are added. One needs to know that the molecular mechanism of the newly added DNA repair ingredient does not compete with those already shown to be synergistic for DNA repair enhancement, namely carotenoids+nicotinamide+zinc.

Nutra-Resveratrol contains the combination of carotenoids+nicotinamide+zinc, which does have prior art, but the addition of Resveratrol material into this composition is novel and unique according to the teaching of U.S. Pat. No. 6,020,351. First of all, the molecular mechanism of Resveratrol has been established in the literature to be cell cycle arrest probably via modulating Sirt 1 (Dario R. Valenzano et al. Current Biology 16 (3): 296-300, 2006; Feng Y H et al. Acta Pharmacol Sin 2002 10: 893-897, 2002; Lorna Whyte et al. Cancer Res 67(24):12007-17, 2007; H. Y. Cohen et al. Science 305 pp. 390-392, 2004). Only recently was it discovered that Resveratrol could modulate DNA repair also. (Gatz, S A et al. Carcinogenesis 2008 29(3):519-527). So at the time of filing the provisional application No. 60/002,314 (Aug. 14, 1995) that gave priority to U.S. Pat. No. 6,020,351 (Feb. 14, 2000) none of the prior on Resveratrol was known or even yet discovered. Now this knowledge is incorporated to support the novelty of the Nutra-Reservatrol application or that revealed in U.S. Pat. No. 6,020,351.

In addition, there has been no prior art occurring since 1995-2000 recognizing the importance of metabolic competition when formulating nutrient compositions for treating age disorders. This is strong evidence it was not obvious to one skilled in the art to practice the principle revealed in U.S. Pat. No. 6,020,351.
2. US patent application 20050196467 (Ser. No. 11/106,200) was published Sep. 8, 2005 and attempts to disclose that carotenoids+nicotinamide+zinc when combined with water soluble extract of Uncaria species and deuterium can be used to improve resistance to DNA damage, enhancing DNA repair and stimulating immune function by treating patients with compositions thereof. I emphasize: The basis of defining prior art to U.S. Pat. No. 6,020,351 by avoiding metabolic competition is not relied upon in this patent application, even though mentioned and cited, but was simply ignored as prior art. Hence, for one skilled in the art there exists no “a priori” reason to justify the utility of why this composition should enhance DNA repair over the composition of carotenoids+nicotinamide+zinc without presentation of data that it actually occurred.
   The Ser. No. 1/106,200 application presents no data whatsoever that deuterium effects DNA repair, nor that Uncaria extracts known to enhance DNA repair as a single ingredient can be combined with carotenoids+nicotinamide+zinc to achieve any additive effects on enhancing DNA repair, are not shown or even contemplated. What is presented is that Uncaria extracts inhibit NF-KB How NF-KB inhibition relates to DNA repair is not known at this time beyond the hypothesis stage. So, empirical data to support DNA repair claims would need to be presented. Again there was none presented to justify using NF-KB instead of DNA repair measurements.
3. US patent application 20050287227 (Ser. No. 11/046,108) was published Dec. 29, 2005. It deals with “administering to a human, or other mammals, a carotenoid material, a nicotinamide material, zinc material, and a water soluble material of a Uncaria species in daily doses effective “in combination to improve the individual resistance to DNA damage, enhance DNA repair capacity, stimulate immune cell function and inhibit tumor cell growth. This patent application recognizes the prior art of combining agents with differing mechanisms of action to achieve synergistic compositions by reference to U.S. Pat. No. 6,020,351 and text comments; it does not supply the enabling the data that demonstrates utility. Unlike Ser. No. 11/106,200, there is data supplied in Ser. No. 11/046,108 that attempts to support its claims, but there are serious defects to note. First of all, only the carotenoids+nicotinamide+zinc combination were shown to enhance DNA repair, already shown with different data in U.S. Pat. No. 6,020,351. The data supporting carotenoids+nicotinamide+zinc+Uncaria species extract can be used is based on NF-KB inhibition on DNA repair enhancement. Although an effective enhanced combination, it is showing that this combination also enhances NF-kB effects not only DNA repair. Hence, the claims are not supported, nor do they teach to one skilled in the art, the data being presented enhance DNA repair or the health benefits being modulated. The NF-kB data mainly support immune and inflammatory responses not DNA repair enhancement. What is specified in the Nutra-Resveratrol application is consistent with U.S. Pat. No. 6,020,351 both the mode of action of Resveratrol as a Sirt 1 modulator of anti-aging effects via cell cycle arrest and direct DNA repair effects have been shown, thus satisfying the criteria for a more effective combination of carotenoids+nicotinamide+zinc when a Resveratrol material was added to the mixture. This is novel to any prior art known at the time of filing U.S. Pat. No. 6,020,351 up to 2009, and it is additionally supported by the fact that there has been no proposed practicing of the invention other that Ser. No. 11/046,200 or Ser. No. 11/046,108.

### Scientific Literature Supporting the Development of Nutra-Resveratrol

### Introduction to Nutra-Resveratrol: By Stuart Garret & Ronald W. Pero

Nutra-Resveratrol is defined herein as a polyphenol (3,5,4′-trihydroxy-stilbene) that can induce cell cycle arrest thereby permitting greater opportunities to remove DNA damage by DNA repair, and thus stimulate normal cell survival and longevity, which in turn can also result in an increased immune cell function (Valenzano, D R et al. Current Biology 16 (3): 296-300, 2006; Feng Y H et al. Acta Pharmacol Sin 2002 10: 893-897,2002; Gatz, S A et al. Carcinogenesis 2008 29(3):519-527). These anti-aging (i.e. DNA repair enhancement) properties for resveratrol materials were not known at the time that combinations of carotenoid-nicotinamide-zinc materials were shown not to metabolically compete with each other, and thus provide an enhanced formulation for delivering aging protection to humans that was first described from 1998-2000 (U.S. Pat. No. 6,020,351, Sheng, Y, et al Cancer Detection and Prevention 1998; 22(4): 284-292). Thus, resveratrol-carotenoid-nicotinamide-zinc compositions are a new discovery now being offered by Stuart Garret and Ronald W. Pero that avoids metabolic competition, a major cause of reduced efficacy via effects on bioavailability, and this knowledge should be added to the previous science already cited herein.

This new improved invention presented by Stuart Garret and Ronald W. Pero relates to a combination of naturally occurring resveratrol material, carotenoid material, nicotinamide material and zinc source material as a combined treatment to aid patients in resisting cellular DNA damage by reducing oxidative cellular damage through enhancing cellular DNA repair capacity (and stimulating immune cellular function) via different modes of action; e.g. cell cycle arrest (resveratrol), oxidative radical scavenger (carotenoids), co-substrate enzymatic stimulator (NAD via nicotinamide), and also a co-factor repair enzyme activator of Poly ADP ribose polymerase (PARP) via its DNA binding zinc finger protein domain. In another specific sense, this, combination of chemicals can be used as a dietary supplementation, or as a drug treatment, to prevent (or improve an individual's ability to resist) DNA damage, enhance DNA repair and stimulate immune function in diseases where these processes are central to the manifested disease state; e.g. ageing, cancer, cardiovascular disease and autoimmune disorders such as diabetes, rheumatoid arthritis and ulcerative colitis.

Stuart Garret and Ronald W. Pero's motivation for this invention is to provide a safe, well-chemically defined, version of resveratrol that would optimize the anti-aging effects when blended into natural products that contain other natural bioactive components that do not metabolically compete, or otherwise modulate bioavailability. In fact, Nutra-Resveratrol is a pharmaceutical-type nutraceutical in that all for ingredients (i.e. resveratrol, carotenoids (canthaxanthin, nicotinamide and zinc salts) are analytical grade being >90% pure. Under these conditions the efficacy standards are defined by known chemical ingredients, and not by undefined or inert supplement component compositions.

Humans have been selected over hundreds of thousands of years to respond to not one chemical but to a myriad of chemicals coming to us through our environment mainly thorough the diet. One can assume that our physiology is extremely well balanced to handle and process these chemical mixtures to extract as efficiently as possible the necessities of life such as nutritional energy sources and chemicals to maintain cellular homeostasis and reproduction. This has to be accomplished without introducing any toxicological consequences. Hence, it follows there is a reasonable likelihood that when humans see natural medicines above the levels normally found in the diet or environment, there exists a strong interaction between the mega doses of natural medicines, so that one supplement limits the uptake and metabolism of another, in an effort to provide a natural selection model by which humans can be protected from the toxicological consequences of overdosing. For example, the practice of prior art teaches that carotenoids and vitamins E or C are all radical (electrophilic) scavengers and that these natural products can be combined into supplements for additive biological effects. However, the literature has not confirmed this practice based on scientific assumption because it was shown that these radical scavengers could inhibit each other's uptake and negate the desired induction of biological effects (Zhang et al., J. Clin. Nutr. 62:1477S-1482S, 1995; Niki et al., Am. J. Clin. Nutr. 62:1322S-1326S, 1995).

The result of Stuart Garret and Ronald W. Pero's latest research efforts are a frontline effort to put forward resveratrol as an anti-aging treatment in the form of a nutritional supplement where its mode of action is optimized. As the anti-aging benefits of resveratrol will no doubt continue to be documented in the literature in the future, it is important to recognize this development innovation that has been based on the assumption that most interventions with resveratrol have been planned and carried out using food, beverages, or undefined crude extracts of plants, where it is well known scientifically that competitive and non-competitive enzymatic inhibition are especially well known to occur. Moreover, these well-known scientific phenomena could seriously alter molecular mechanisms governed by substrate/product levels, which are in turn under regulation of competitive metabolism.

It is to be understood that the invention is not limited to the features and embodiments hereinabove specifically set forth, but may be carried out in other ways without departure from its spirit.

What is claimed is:

1. An improved composition of matter for administration to humans or other animals, consisting essentially of a combination of resveratrol material, carotenoid material, nicotinamide material and zinc source material and essentially free of other active ingredients.

2. An improved composition as defined in claim 1, in a formulation for oral administration.

3. An improved composition as defined in claim 1, in a formulation for parenteral administration.

4. An improve composition as defined in claim 1, wherein said carotenoid material is selected from the group consisting of alpha carotene, beta carotene, canthaxanthin, lycopene and mixtures thereof.

5. An improved composition as defined in claim 1, wherein said nicotinamide material is selected from the group consisting of nicotinamide, niacin, tryptophan and mixtures thereof.

6. An improved composition as defined in claim 1, wherein said zinc source material is one or more zinc salts.

7. An improved composition as defined in claim 1, wherein said resveratrol material is a polyphenol (e.g., 5,4′-trihydroxy-stilbene also known as a phytolexin or phytoestrogen) that modulates cell cycle events governing growth arrest.

8. An improved composition as defined in claim 4, wherein said carotenoid material is selected from the group consisting of alpha carotene, beta carotene, canthaxanthin, lycopene and mixtures thereof, and said nicotinamide material is selected from the group consisting of nicotinamide, niacin and tryptophan and mixtures thereof.

9. An improved composition as defined in claim 1, wherein said resveratrol material, carotenoid material, nicotinamide material and zinc source material are present in proportions effective, in said combination, to improve resistance to DNA damage, enhance DNA repair capacity, and stimulate immune function in a human subject to whom the composition is administered as a daily dosage.

10. An improved method of treating a human or other animal subject, consisting of the steps of
(a) administering resveratrol, carotenoid material, nicotinamide material and zinc source material to the subject to selectively supplement the subject's dietary intake thereof and
(b) repeating step (a) substantially daily.

11. An improved method according to claim 9, wherein about 1500-6000 mg resveratrol material, about 100-200 mg of carotenoid material, about 100-200 mg of nicotinamide material and about 10-20 mg of zinc source material are administered in step (a).

12. An improved method of treating a human subject consisting of selectively administering to the subject resveratrol material, carotenoid material, nicotinamide material and zinc source material, said materials being administered to the subject in daily dosage amounts effective, in combination, to improve resistance to DNA damage, enhance DNA repair capacity, and stimulate immune function.
